(19) United States
(12) Patent Application Publication
(10) Pub. No.: US 2018/0364024 A1
(43) Pub. Date: Dec. 20, 2018

(54) BEAM-SHAPING ELEMENTS FOR OPTICAL COHERENCE TOMOGRAPHY PROBES

(71) Applicant: Corning Incorporated, Corning, NY (US)

(72) Inventors: Adra Smith Baca, Hickory, NC (US); Robert Randall Hancock, JR., Corning, NY (US); Gary Allen Hart, Walworth, NY (US); Horst Schreiber, Livonia, NY (US); Daniel Max Staloff, Rochester, NY (US); Jue Wang, Fairport, NY (US)

(21) Appl. No.: 15/736,937

(22) PCT Filed: Jun. 17, 2016
(86) PCT No.: PCT/US2016/037957

§ 371 Date: Dec. 15, 2017

Related U.S. Application Data
(60) Provisional application No. 62/180,717, filed on Jun. 17, 2015.

(51) Int. Cl. G01B 9/02; A61B 5/00; G02B 27/09

(52) U.S. Cl. CPC G01B 9/0205; G01B 9/02091; G01B 9/02034; A61B 2562/0233; A61B 5/0084; G02B 27/0983; A61B 5/0066

(57) ABSTRACT

A beam-shaping optical system suitable for use with optical coherence tomography having a beam-shaping insert having a polymeric material, the beam-shaping insert integrally defining a beam-shaping element. The beam-shaping element has a reflective element positioned on a curved surface. A light source generates an electromagnetic beam. An optical fiber having a core and a cladding, the optical fiber having first end optically coupled with the light source and a fiber end. The fiber end is configured to emit the electromagnetic beam toward the beam-shaping element. The reflective element has a reflectivity greater than about 98% for both a first wavelength band of the electromagnetic beam and a second wavelength band of the electromagnetic beam.

This application claims the benefit of priority under 35 U.S.C. § 119 of U.S. Provisional Application Ser. No. 62/180,717 filed on Jun. 17, 2015, the content of which is relied upon and incorporated herein by reference in its entirety.

### BACKGROUND

The present disclosure relates to optical coherence tomography, and in particular, to beam-shaping elements for an optical coherence tomography probe.

Optical coherence tomography (OCT) is used to capture a high-resolution cross-sectional image of biological tissues and is based on fiber-optic interferometry. The core of an OCT system is generally known as a Michelson interferometer, which typically includes a first optical fiber which is used as a reference arm and a second optical fiber which is used as a sample arm. The sample arm includes the sample to be analyzed, as well as a probe that contains optical components therein. A light source upstream of the probe provides light used in imaging. A photodetector is arranged in the optical path downstream of the sample and reference arms. The probe is used to direct light into or onto the sample and then to collect scattered light from the sample.

Optical interference of light from the sample arm and the reference arm is detected by the photodetector only when the optical path difference between the two arms is within the coherence length of the light from the light source. Depth information from the sample is acquired by axially varying the optical path length of the reference arm and detecting the interference between light from the reference arm and scattered light from the sample arm. A three-dimensional image is obtained by transversely scanning in two dimensions the optical path in the sample arm. The axial/depth resolution of the process is determined by the coherence length, while the overall transverse resolution is dictated by the size of the image spot formed by the optical components of the probe.

Because the probe typically needs to be inserted into a small cavity of the body, generally it must be small and preferably have a simple optical design. Exemplary designs for the probe include a transparent cylinder in which the miniature probe optical components are contained and through which light is transmitted and received. However, light may be lost due to back reflection when it passes through materials having a different refractive index, thus decreasing image spot intensity. Additionally, back reflections decrease the signal to noise ratio in the data. Moreover, having multiple and separate optical components in the probe is generally problematic because the small optical components have to be assembled and aligned, which adds to the cost and complexity of manufacturing the probe.

### SUMMARY

According to one embodiment of the present disclosure, a beam-shaping optical system suitable for use with optical coherence tomography having a beam-shaping insert having a polymeric material, the beam-shaping insert integrally defining a beam-shaping element. The beam-shaping element has a reflective element positioned on a curved surface. A light source generates an electromagnetic beam. An optical fiber having a core and a cladding, the optical fiber having first end optically coupled with the light source and a fiber end. The fiber end is configured to emit the electromagnetic beam toward the beam-shaping element. The reflective element has a reflectivity greater than about 98% for both a first wavelength band of the electromagnetic beam and a second wavelength band of the electromagnetic beam.

According to another embodiment of the present disclosure, an optical coherence tomography probe has a sheath defining a central cavity, a beam-shaping insert positioned in the central cavity, the insert having a polymeric material and defining a curved surface, and a reflective element positioned on the curved surface. The reflective element includes a barrier layer having at least one layer of aluminum, chromium or alumina positioned on the curved surface. A metal layer is positioned on the barrier layer. At least one stack of alternating dielectric materials is positioned on the metal layer. A ferrule is positioned within the central cavity. An optical fiber, the fiber supported by the ferrule including a fiber end configured to emit an electromagnetic beam toward the reflective element.

According to another aspect of the present disclosure, a method of forming an optical coherence tomography probe includes the steps of forming a polymeric beam-shaping insert defining a curved surface, depositing a barrier layer on the curved surface, the barrier layer comprising at least one layer of chromium, aluminum, and alumina, depositing a metallic layer on the barrier layer, and depositing a dielectric stack on the metallic layer to form a reflective element. The reflective element is configured to reflect greater than about 98% of both a first wavelength band of an electromagnetic beam and a second wavelength band of an electromagnetic beam.

According to another aspect of the present disclosure, a beam-shaping optical system suitable for use with optical coherence tomography includes a sheath defining a central cavity, a beam-shaping insert having a first beam-shaping element and a second beam-shaping element, the insert positioned within the cavity, and an optical fiber having a core and a cladding disposed within the central cavity. The optical fiber has a fiber end configured to emit an electromagnetic beam toward the beam-shaping insert. The first beam-shaping element reflects a first portion of the electromagnetic beam and the second beam-shaping element refracts a second portion of the electromagnetic beam.

According to another aspect of the present disclosure, an optical coherence tomography probe includes a sheath defining a central cavity, a beam-shaping insert positioned near an end of the central cavity, a beam-shaping element positioned on the beam-shaping insert, and an optical fiber having a core and a cladding disposed within the central cavity. The optical fiber has a fiber end configured to emit an electromagnetic beam toward the beam-shaping element. The beam-shaping element is configured to focus a first portion of the electromagnetic beam to a side of the sheath and focus a second portion of the electromagnetic beam forward of the sheath.

According to another aspect of the present disclosure, a method of forming multiple image spots includes the steps of positioning an optical fiber having a core and a cladding within a ferrule, positioning the ferrule within a central cavity of a sheath, and emitting an electromagnetic beam from a fiber end of the optical fiber toward a beam-shaping insert. The beam-shaping insert is configured to form a first image point at a first image plane and a second image point at a second image plane, the image planes being different working distances from the beam-shaping insert.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1A is an elevated exploded view of an optical probe for use in OCT according to one embodiment;

FIG. 1B is an elevated perspective cross-sectional view of the optical probe depicted in FIG. 1 in assembly taken at line IB-IB of FIG. 1A according to one embodiment;

FIG. 2 is a partially enlarged cross sectional view taken at section II of FIG. 1B;

FIG. 3 is a partially enlarged cross sectional view of the optical probe taken at line IB-IB of FIG. 1A according to one embodiment;

FIG. 4A is a partially enlarged cross sectional view of the optical probe taken at line IB-IB of FIG. 1A according to one embodiment;

FIG. 4B is a partially enlarged cross sectional view of the optical probe taken at line IB-IB of FIG. 1A according to another embodiment;

FIG. 4C is a partially enlarged cross sectional view of the optical probe taken at line IB-IB of FIG. 1A according to yet another embodiment;

FIG. 4D is a partially enlarged cross sectional view of the optical probe taken at line IB-IB of FIG. 1A according to yet another embodiment;

FIG. 5 is a schematic diagram of an OCT alignment system that includes the optical probe according to one embodiment;

FIG. 6 is a schematic diagram of an OCT system that includes the optical probe according to one embodiment;

FIG. 7A is a graph depicting the reflectance of an optical probe reflective element made according to an aspect of this disclosure;

FIG. 7B is a bar chart depicting the thickness of an optical probe reflective element made according to an aspect of this disclosure;

FIG. 8A is a graph depicting the reflectance of an optical probe reflective element made according to another aspect of this disclosure; and

FIG. 8B is a bar chart depicting the thickness of an optical probe reflective element made according to another aspect of this disclosure.

### DETAILED DESCRIPTION

Reference will now be made in detail to the present preferred embodiments, examples of which are illustrated in the accompanying drawings. Whenever possible, the same reference numerals will be used throughout the drawings to refer to the same or like parts.

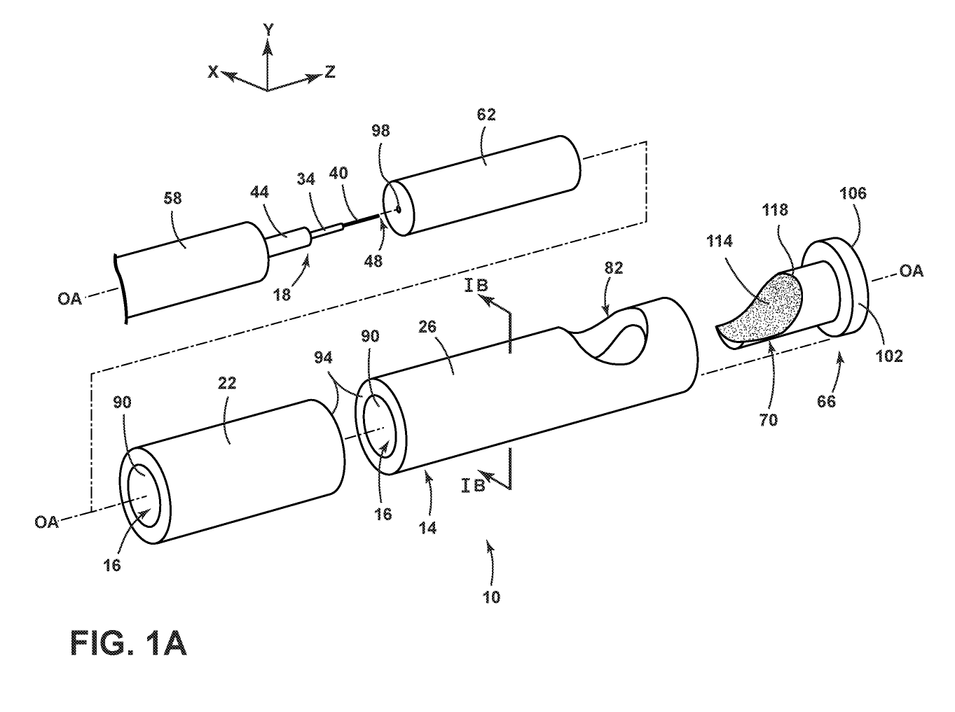

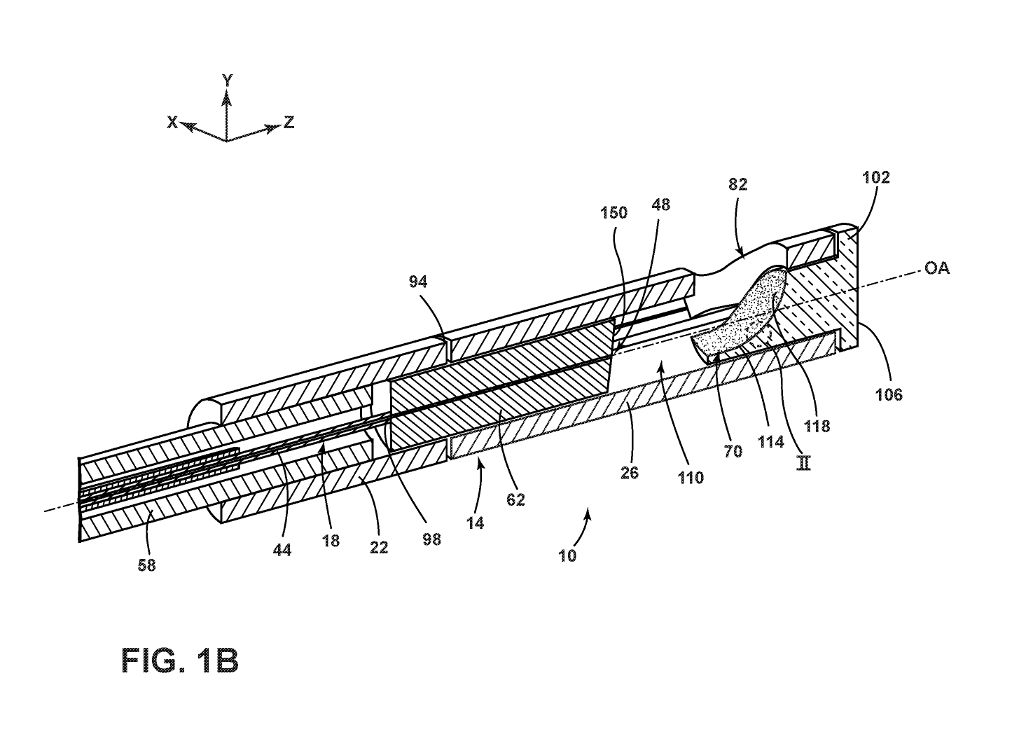

For purposes of description herein, the terms “upper,” “lower,” “right,” “left,” “rear,” “front,” “vertical,” “horizontal,” and derivates thereof shall relate to an optical probe 10 as oriented in FIG. 1A, unless stated otherwise. However, it is to be understood that the optical probe 10 may assume various alternative orientations, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Depicted in FIGS. 1A-6 is an embodiment of the beam-shaping optical probe 10 suitable for use in OCT and the making of OCT images. The optical probe 10 includes a sheath 14 defining a central cavity 16 within which an optical fiber 18 is disposed. The sheath 14 is comprised of a first portion 22 and a second portion 26. The optical fiber 18 includes a cladding 34, a core 40, and a coating 44. In various embodiments the coating 44 is polymeric, but may also comprise metal. The optical fiber 18 includes a first end (not shown) optically coupled to a light source (not shown) and a fiber end 48. The light source is configured to generate and emit an electromagnetic beam 52 into the optical fiber 18 such that the fiber end 48 emits the electromagnetic beam 52. The electromagnetic beam 52 may be a light beam (e.g., visible, ultraviolet, infrared or light). The electromagnetic beam 52 is emitted along an optical axis OA defined by the optical probe 10. In assembly, the optical fiber 18 enters the optical probe 10 through a torque tube 58 and is coupled to a ferrule 62. A beam-shaping insert 66 is positioned at a distal end of the optical probe 10 and defines a beam-shaping element 70.

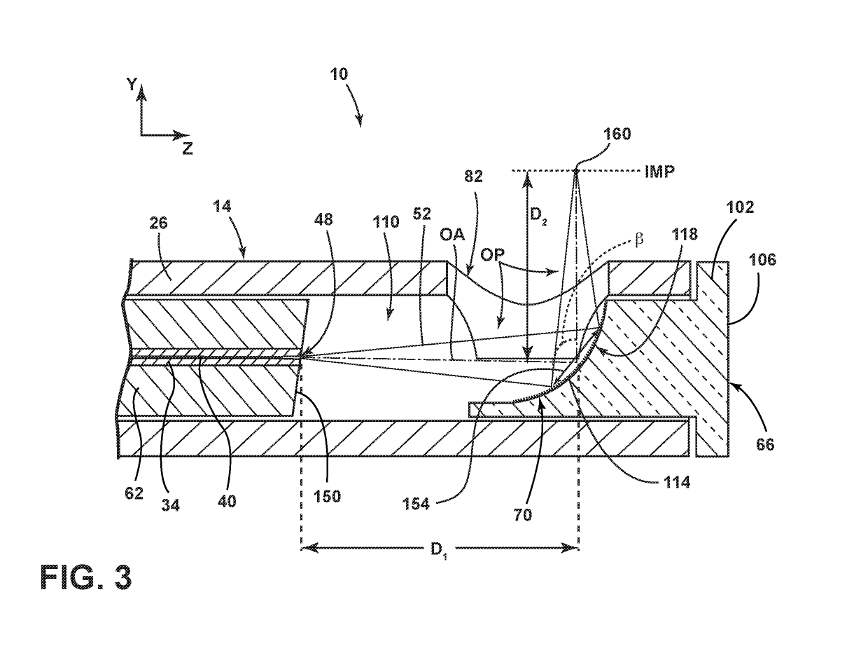

Referring now to FIGS. 1A and 1B, the sheath 14 is an assembly of the first portion 22 and the second portion 26 aligned on axis OA and in abutment with one another. In the depicted embodiment, the second portion 26 defines a window 82 through which the electromagnetic beam 52 (FIG. 3) may exit and enter the optical probe 10. Optionally, the window 82 may include a transparent material through which the electromagnetic beam 52 can pass, yet prevents foreign matter out of the optical probe 10. The sheath 14 may comprise a transparent or opaque material. In some embodiments the sheath 14 may comprise a polymeric material such as latex, polyethylene, or polyurethane or a metal such as 304 or 306 stainless steel. The central cavity 16 of the sheath 14 is defined by an inner wall 90. The first and second portions 22, 26 each define an abutment surface 94 configured to be in contact or close proximity when the optical probe 10 is in the assembled configuration. The ferrule 62, the torque tube 58 and the beam-shaping insert 66 are shaped to precisely mirror the inner wall 90 of the sheath 14 such that the ferrule 62, torque tube 58 and the beam-shaping insert 66 precisely fit within the central cavity 16 in a flush and substantially concentric manner. In assembly, the optical fiber 18 travels through the torque tube 58 from an upstream light source (not shown) to the ferrule 62. The ferrule 62 defines an aperture 98 extending though the ferrule 62 into which the optical fiber 18 is positioned. The aperture 98 is configured to accept the cladding 34 and the core 40 of the optical fiber 18. By positioning the optical fiber 18 within the ferrule 62, a central axis of the fiber 18 along which the electromagnetic beam 52 is emitted may be quickly aligned to the optical axis OA of the optical probe 10 due to the high concentricity between the ferrule 62 and the inner wall 90 of the sheath 14.

The beam-shaping insert 66 is configured to be inserted into the central cavity 16 of the distal end of the sheath 14 such that a flange 102 is in abutting contact with the sheath 14. It will be understood that various embodiments of the optical probe 10 and beam-shaping insert 66 do not necessarily have a flange 102. The flange 102 is positioned on the beam-shaping insert 66 such that the flange 102 contacts the second portion 26 of the sheath 14 as the beam-shaping element 70 is positioned proximate the window 82. In this manner, the flange 102 may aid in the positioning of the beam-shaping insert 66 within the sheath 14 as well as the beam-shaping element 70. Optionally, a forward surface 106 of the beam-shaping insert 66 and/or the flange 102 includes one or more markings (e.g., degree dial, an index line, hash marks) designed to aid an operator in correctly orienting the beam-shaping insert 66 within the sheath 14. Additionally or alternatively, the sheath 14 (e.g., second portion 26) may include the same, similar, or complimentary markings configured to aid in orientation of the beam-shaping insert 66. Orientation of the beam-shaping insert 66 within the sheath 14 is performed such that the beam-shaping element 70 is aligned with the optical axis OA of the optical probe 10 and the window 82 of the sheath 14. A gap 110 is defined between the ferrule 62 and the beam-shaping insert 66 when in assembly. The gap 110 may be a void having air or a transmissive liquid or solid. In embodiments where the gap 110 is filled with a transmissive liquid or solid, the refractive index of the liquid or solid may be chosen to aid in propagation and/or shaping of the electromagnetic beam 52.

In various embodiments, the beam-shaping insert 66 and/or the ferrule 62 includes a polymeric composition having a glass transition temperature greater than about 150° C. Exemplary thermoset classes of polymeric materials that may be used to form the beam-shaping element 66 include epoxy, polyester, cyanate ester, phenolic, melamine, bismalemide, and polyimide. Exemplary thermoplastic polymeric materials for the beam-shaping insert 66 include ZEONOR® (available from Zeon Chemicals L.P., Louisville, Ky.), polyetherimide (PEI), polyethylene, polypropylene, polycarbonate, engineered polymers (e.g., liquid crystal), acrylonitrile butadiene styrene, polyetheretherketone, nylon 12, polybutylene terephthalate, polyethylene terephthalate, polysulfones, thermoplastic polyimide, cyclo olefinic copolymer, polyphenylene ether, polyphenylene sulfide, syndiotactic polystyrene, as well as any other polymeric material or combination of polymeric materials capable of forming the beam-shaping insert 66 and producing a smooth surface. In polymeric embodiments, the beam-shaping insert may also include filler including mineral fillers, glass fibers, or a combination of mineral and glass fibers. In other embodiments, the beam-shaping insert 66 may include metals, ceramics, or composites thereof. The beam-shaping insert 66 and/or the ferrule 62 is capable of formation by conventional manufacturing techniques such as injection molding, casting, machining, thermoforming, diamond turning, or extrusion.

Still referring to FIGS. 1A and 1B, the beam-shaping element 70 is integrally defined by the beam-shaping insert 66 such that in assembly, the beam-shaping element 70 is positioned inside of the central cavity 16 of the sheath 14. The beam-shaping element 70 includes a reflective element 114 positioned on a curved surface 118 defined from the beam-shaping insert 66. The beam-shaping insert 66 extends in an upwardly and inwardly curved manner with respect to the forward surface 106 to define the curved surface 118. The beam-shaping element 70 is substantially conic in shape and curves inwardly toward the optical axis OA of the optical probe 10. The conic shape of the beam-shaping element 70 is defined by a radius of curvature and conic constant along an axis of the beam-shaping element 70 with respect to the optical axis OA of the optical probe 10.

In order to properly shape the electromagnetic beam 52, the beam-shaping element 70 may have a radius of curvature along the X-axis that is the same or different than a radius of curvature in the Y-axis. The radius of curvature of the X- and Y-axes of the curved surface 118 of the beam-shaping element 70 may have an absolute value of between about 0.5 millimeters and about 10 millimeters, and more specifically, about 1.0 millimeter to about 4.0 millimeters. The conic constant of the X- and Y-axes of the beam-shaping element 70 may independently range from about 1 to about −2, and more specifically between about 0 and about −1. It should be understood that the radii and conic constants of the curved surface 118 explained above describe the overall shape of the beam-shaping element 70, and do not necessarily reflect local radii or conic constants of the curved surface 118. The radius of curvature of the X-axis and Y-axis of the beam-shaping element 70 may be adjusted independently in order to correct for any material disposed around the optical probe 10. The conic shape of the beam-shaping element 70 may be decentered along the Y- or Z-axes between about 0.01 millimeters and about 0.8 millimeters. Additionally, the conic shape of the beam-shaping element 70 may have a rotation between the Y- and Z-axes of between about 70° and 120°.

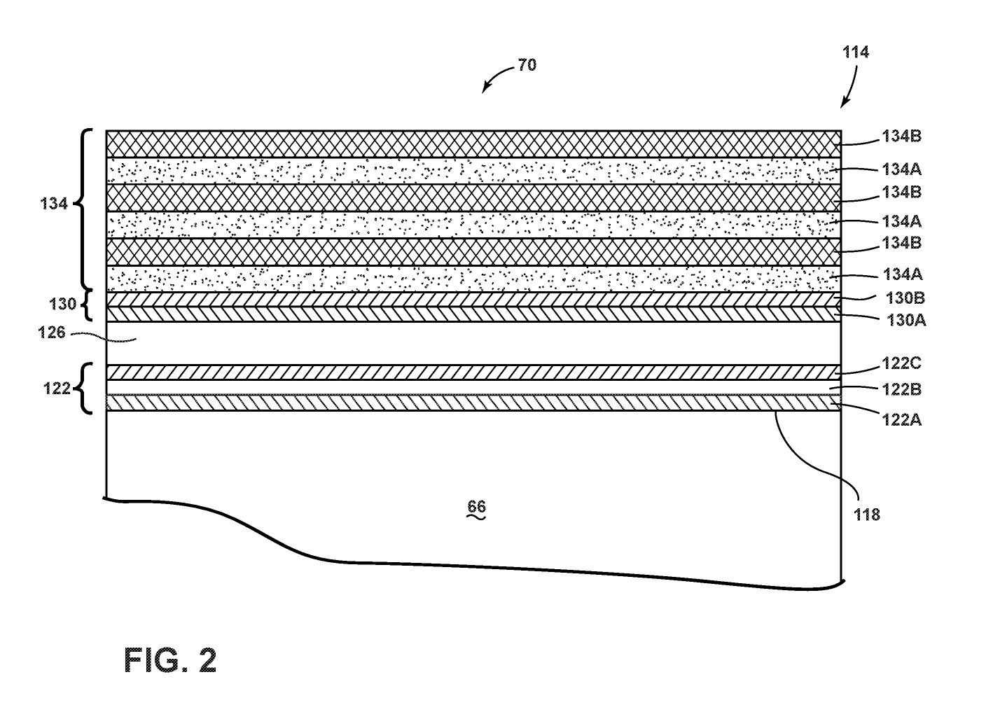

Referring now to FIG. 2, the beam-shaping element 70 is configured to collect and shape (e.g., collimate, converge, and/or change the optical path of) through reflection the electromagnetic beam 52 (FIG. 3) emitted from the optical fiber 18, as explained in greater detail below. Positioned on the curved surface 118 of the beam-shaping element 70 is the reflective element 114. In the depicted embodiment, the reflective element 114 includes a barrier layer 122, a metal layer 126, a first dielectric sack 130 and a second dielectric stack 134. The barrier layer 122 comprises a chromium layer 122A, an aluminum layer 122B, and an alumina layer 122C. In some embodiments, the barrier layer 122 includes only one or two of the layers 122A, 122B, 122C (e.g., only the chromium layer 122A or only the aluminum layer 122B and the alumina layer 122C). The order of the layers 122A, 122B, 122C may also be different than that depicted. For example, the alumina layer 122C may be proximate the curved surface 118 of the aluminum layer 122B may be proximate the metallic layer 126. Each of the layers 122A, 122B, 122C may have a thickness between about 1 nanometers and about 100 nanometers, more particularly between about 10 nanometers and about 60 nanometers, and more particularly about 20 nanometers to about 40 nanometers. In a specific example, the layers 122A, 122B, 122C are each about 30 nanometers thick. In some embodiments, the thickness of the layers 122A, 122B, 122C may all be approximately the same, while in other embodiments each layer 122A, 122B, 122C may have a different thickness. The chromium layer 122A may include metallic chromium, alloys of chromium, oxides of chromium, or high chromium concentration (e.g., greater than about 30 weight %) materials. Similarly to the chromium layer 122a, the aluminum layer 122B may include metallic aluminum, oxides of aluminum, aluminum alloys, and high aluminum concentration (e.g., greater than about 30 weight %) materials. The alumina layer 122C may include various oxides of aluminum, metallic aluminum, aluminum alloys, and high alumina concentration (e.g., greater than about 30 weight %) materials and other metal oxides. The chromium layer 122A, aluminum layer 122B and the alumina layer 122C of the barrier layer 122 may be sprayed, dipped, spun, or brushed onto the curved surface 118 of the beam-shaping insert 66.

Traditional applications utilizing a metal (e.g., metal layer 126) or dielectric stack as a beam-shaping element 70 on a polymeric component (e.g., beam-shaping insert 66) suffer from low adhesion strength and are prone to chipping or peeling off. However, application of the barrier layer 122 to the curved surface 118 of the beam-shaping insert 66 offers several advantages over simply applying the metal layer 126 or the first and second dielectric stacks 130, 134 directly to the curved surface 118. The barrier layer 122 may increase the adhesion strength with which the metal layer 126 is held to the curved surface 118. For example, use of the barrier layer 122 may allow the metal layer 126 and the first and second dielectric stacks 130, 134 to survive military specification adhesion requirements (e.g., a ½″ wide strip of cellophane tape is pressed against the reflective element 114 and quickly removed). Additionally, the use of the barrier layer 122 may prevent the transfer of thermal energy to the beam-shaping insert 66 from the electromagnetic beam 52 during beam-shaping thus preventing possible damage from occurring to the beam-shaping insert 66 or element 70.

Positioned on top of the barrier layer 122 is the metal layer 126. The metal layer 126 may have a thickness from about 50 nanometers to about 200 nanometers, or from about 75 nanometers to about 150 nanometers, or from about 80 nanometers to about 120 nanometers. In a specific embodiment, the metal layer 126 is about 100 nanometers thick. The metal layer 126 may include silver, gold, aluminum, platinum, copper, alloys thereof and other lustrous metals capable of reflecting the electromagnetic beam 52. In various embodiments, the metal layer 126 may be applied via physical vapor deposition or by spray coating. Use of the metal layer 126 offers a general broadband reflection to the reflective element 114.

Positioned above the metal layer 126 are the first and second dielectric stacks 130, 134. It should be understood that although depicted with two dielectric stacks, the reflective element 114 may have only one stack (e.g., the first or second dielectric stacks 130, 134) or have three or more stacks. The first dielectric stack 130 is positioned on the metal layer 126 and includes at least one first dielectric layer 130A and at least one second dielectric layer 130B. The first dielectric stack 130 may contain between two and ten layers (e.g., the first and second dielectric layers 130A, 130B). The first and second dielectric layers 130A, 130B are positioned in an alternating manner and comprise a dielectric material. Exemplary dielectric materials include SiO₂, Ta₂O₅, NbO₅, TiO₂, HfO₂, and combinations thereof. In some embodiments, each layer 130A, 130B may be a single dielectric material. In a specific embodiment, the first dielectric layer 130A may be SiO₂ and the second dielectric layer 130B may be Ta₂O₅. The thickness of the first and second dielectric layers 130A, 130B may be between about 50 nanometers and about 500 nanometers. In some embodiments, the thickness of the first and second dielectric layers 130A, 130B may be different than one another and optionally vary across the thickness of the first dielectric stack 130. In some embodiments, the choice of which dielectric material to use for the alternating first and second dielectric layers 130A, 130B may be based on the refractive index of the material in order to increase a reflectivity of the reflective element 114. For example, a high refractive index material (e.g., Ta₂O₅, NbO₅, TiO₂, HfO₂) may be included in the first dielectric layer 130A and a low refractive index material (e.g., SiO₂) may be included in the second dielectric layer 130B. In some embodiments, the upper most layer (e.g., first or second dielectric layer 130A, 130B) comprises a high refractive index material (e.g., Ta₂O₅, NbO₅, TiO₂, HfO₂). Additionally or alternatively, the upper most layer may be thinner (e.g., half or quarter the thickness of the wavelength of the beam 52) or thicker than the other layers (e.g., first or second dielectric layers 130A, 130B).

Similarly to the first dielectric stack 130, the second dielectric stack 134 also includes alternating layers of dielectric materials. In the depicted embodiment, the second dielectric stack 134 includes at least one third dielectric layer 134A and at least one fourth dielectric layer 134B. The second dielectric stack 134 may contain between two and ten layers (e.g., the third and fourth dielectric layers 134A, 134B). The third and fourth dielectric layers 134A, 134B of the second dielectric stack 134 may comprise any of the dielectric materials and have any of the thicknesses mentioned in connection with the first dielectric stack 130. In some embodiments, the thickness or ratio of thicknesses of the third and fourth dielectric layers 134A, 134B may be different (e.g., smaller or larger) than that of the first or second dielectric layers 130A, 130B of the first dielectric stack 130. It will be understood that more than two types of layers may be used in the construction of the reflective element 114. As explained in connection with the first dielectric stack 130, the material chosen for the third and fourth dielectric layers 134A, 134B may be chosen based on index of refraction in order to increase reflectivity of the reflective element 114. Further, in embodiments utilizing the second dielectric stack 134, an uppermost layer of the stack may be thicker or thinner than the rest of the layers (e.g., third or fourth dielectric layers 134A, 134B). Additionally, it will be understood that dielectric materials having a suitable index of refraction not specified here may be used with a variety of thicknesses in order to approximate the dielectric materials disclosed in connection with the first and second dielectric stacks 130, 134.

Use of dielectrics (e.g., the first and/or second dielectric stacks 130, 134) within the reflective element 114 may allow the beam-shaping element 70 to be a dual-channel beam-shaping element 70. In such an embodiment, the reflective element 114 may have a reflectivity of greater than about 98% for two different wavelength bands of the electromagnetic beam 52. For example, the two different wavelength bands may be an imaging band and a high power band. In such embodiments, the imaging band of the electromagnetic beam 52 may have a wavelength of between about 700 nanometers and about 830 nanometers, or between about 1200 nanometers to about 1400 nanometers. Imaging wavelength bands of the electromagnetic beam 52 may be useful for the formation of images using the optical probe 10. High power bands of the electromagnetic beam 52 may have a wavelength of between about 1430 nanometers and about 1550 nanometers. High power bands of the electromagnetic beam 52 may also cover water absorption spectrums. High power bands of the electromagnetic beam 52 may be useful in the optical probe 10 for marking or ablation purposes. During operation at the high power bands, the electromagnetic beam 52 may have a peak intensity as measured at the beam-shaping element 70 of between about 500 watts per square centimeter to about 15,000 watts per square centimeter, or from about 1,000 watts per square centimeter to about 11,000 watts per square centimeter. In a specific example, the electromagnetic beam power may be about 8,000 watts per square centimeter as measured at the beam-shaping element 70. The reflectance of the reflective element 114 may vary based on the angle of incidence of the electromagnetic beam 52 on the element 114. The reflective element 114 may also include a capping layer to protect it from environmental conditions (e.g., water, oxygen, and/or sterilization procedures).

Referring now to FIG. 3, the optical fiber 18 is depicted as defining the fiber end 48 flush with a face 150 of the ferrule 62. In operation, the optical fiber 18 is configured to act as a wave guide for electromagnetic radiation, specifically light at an operating wavelength λ. The optical fiber 18 carries light from an upstream light source (not shown) to the fiber end 48 where the light is emitted as the electromagnetic beam 52. In one embodiment, the operating wavelength λ includes an infrared wavelength such as one in the range from about 830 nanometers to about 1,600 nanometers, with exemplary operating wavelengths λ being about 1300 nanometers and about 1560 nanometers. In various embodiments, the operating wavelengths λ may be as low as about 700 nanometers. The optical fiber 18 may be a single mode or a multimode configuration. The optical fiber 18 may have a mode field diameter of between about 9.2 microns+/−0.4 microns at a wavelength of 1310 nanometers and have a mode field diameter of about 10.4 microns+/−0.5 microns at 1550 nanometers. The diameter of the cladding 34 may be between about 120 microns and about 130 microns.

The ferrule 62 is configured to couple with the inner wall 90 of the sheath 14 such that when the optical fiber 18 is within the aperture 98, the electromagnetic beam 52 is emitted from the fiber end 48 on an optical path OP that is both substantially coaxial with the optical axis OA of the optical probe 10, and directed toward the beam-shaping element 70. As the beam 52 is emitted from the fiber end 48, it propagates through the gap 110 and the diameter of the optical path OP widens with increasing distance from the fiber end 48. A distance D₁ between the fiber end 48 and the reflective element 114 of the beam-shaping element 70 is set based on a desired size of a beam spot 154. The beam spot 154 is the area of light the electromagnetic beam 52 forms as it strikes the beam-shaping element 70. The beam spot 154 grows in diameter with increasing distance D₁ from the fiber end 48. In order for the beam-shaping element 70 to properly shape the electromagnetic beam 52, the beam spot 154 must be have the proper diameter when contacting the reflective element 114 (e.g., approximately half the diameter of the reflective element 114). Accordingly, the ferrule 62 and the fiber end 48 must be placed a predetermined distance from the beam-shaping element 70 for the beam 52 to be properly shaped. In various embodiments, the distance D₁ between the fiber end 48 and the reflective element 114 may range between about 0.2 millimeters and about 2.6 millimeters. In one embodiment, the distance D₁ is about 1.314 millimeters. The diameter of the beam spot 154 may range from about 200 microns to about 2000 microns and more specifically, between about 400 microns to about 600 microns.

As the electromagnetic beam 52 enters the beam-shaping element 70, its optical path OP is folded by an angle β from reflection off of the reflective element 114. In the depicted embodiment, the angle β is approximately 90°, but in various embodiments can vary greater than or less than about 25°, about 20°, and about 10° on either side of 90°. The radius of curvature and position of the beam-shaping element 70 determine both the angle β that the optical path OP of beam 52 will be folded by, and also a working distance D₂ to an image plane IMP where the beam 52 converges to form an image spot 160. Accordingly, the emitted beam 52 is shaped into the image spot 160 solely by reflection from the beam-shaping element 70.

Still referring to FIG. 3, the fiber end 48 of the optical fiber 18 may terminate at an angle in order to prevent undesired back reflection of light into the fiber 18. OCT is particularly sensitive to back reflections of light which have not been scattered off of a sample to be tested (i.e., reflections from the optical probe 10, fiber end 48, or refractive surfaces along the optical path OP). The back reflected light may lead to increased noise and artifacts in the OCT image. Terminating the fiber end 48 at an angle minimizes the coupling of the back reflected light back into the optical fiber 18. The fiber end 48 may be prepared at an angle between about 0° to about 10°, and more particularly between about 6° to 9°. Angling of the fiber end 48 may be accomplished, for example, by cleaving the fiber end 48 before or after insertion into the ferrule 62, or by polishing the face 150 of the ferrule 62 with the fiber end 48 at an angle, as depicted. In some embodiments, the ferrule 62 or beam-shaping element 70 may be angled with respect to the optical axis OA of the optical probe 10 in order to compensate for the angled fiber end 48. The angled ferrule 62 would keep the optical path OP of the beam 52 substantially coaxial with the optical axis OA of the optical probe 10. Additionally or alternatively, the fiber end 48 may include an anti-reflection film to reduce the amount of reflected light absorbed by the optical fiber 18. The anti-reflection film may include a single or multilayer dielectric material configured to cancel light reflected back to the optical probe 10.

In various embodiments, the fiber end 48 of the optical fiber 18 may be locally tapered with respect to the rest of the optical fiber 18. Tapering of the fiber end 48 may be accomplished through laser heating, plasma heating, resistance heating, or flame heating a portion of the optical fiber 18, and placing the fiber 18 in tension. The heated portion of the fiber 18 then necks down as it is pulled. The fiber 18 may be pulled until the fiber 18 is separated or the heated portion of the fiber 18 may be cut while in the necked down position. Tapering of the core 40 may have an axial length along the optical fiber 18 of about 1 millimeter to about 5 millimeters, and in a specific example of about 4 millimeters. The tapering of the fiber end 48 should be such that the fiber end 48 does not experience adiabatic loss. Tapering of the optical fiber 18 at the fiber end 48 may locally increase the mode field diameter of the fiber end 48. The mode field diameter at a beam 52 wavelength of 1310 nanometers of the tapered fiber end 48 may range from about 10 microns to about 40 microns and in specific examples be about 10 microns, about 11 microns, about 12 microns, about 13 microns, about 14 microns, about 15 microns, about 16 microns, about 17 microns, about 18 microns, about 19 microns, or about 20 microns. The mode field diameter of the fiber end 48 may expand about 5%, about 10%, about 100%, about 400%, or about 500%. Tapering of the optical fiber 18 at the fiber end 48 may locally increase the mode field diameter of the fiber end 48. The mode field diameter at a beam 52 wavelength of 1310 nanometers of the tapered fiber end 48 may range from about 10 microns to about 40 microns and in specific examples be about 10 microns, about 11 microns, about 12 microns, about 13 microns, about 14 microns, about 15 microns, about 16 microns, about 17 microns, about 18 microns, about 19 microns, or about 20 microns. Tapering and angling the fiber end 48 of the optical fiber 18 may decrease the back reflection from about −10 dB to about −350 dB, and in specific examples to below about −80 dB, −90 dB, −100 dB, −110 dB, −120 dB and below about −130 dB depending on the level of tapering. Additionally or alternatively, the fiber end 48 may be tapered and positioned at locations other than at the face 150 of the ferrule 62. For example, a second optical fiber having similar dimensions to that of the tapered fiber end 48 may be positioned in the aperture 98 of the ferrule 62 and be optically coupled to the fiber end 48. In such embodiments, the optical coupling may take place at any point along the aperture 98 (e.g., inside the ferrule 62) as well as at the entrance to the aperture 98. The second optical fiber may then have an angled end, from which the electromagnetic beam 52 exits, to reduce back reflection.

In other embodiments, the core 40 of the fiber end 48 may be locally expanded in addition to being prepared with an angle. The core 40 of the optical fiber 18 may be locally expanded at the fiber end 48 such that the mode field diameter of the fiber 18 locally increases. In expanded core 40 embodiments, the fiber end 48 may have a mode field diameter at a beam 52 wavelength of 1310 nanometers between about 10 microns to about 40 microns with specific examples being about 10 microns, about 11 microns, about 12 microns, about 13 microns, about 14 microns, about 15 microns, about 16 microns, about 17 microns, about 18 microns, about 19 microns, and about 20 microns. The mode field diameter and diameter of the core 40 of the fiber end 48 may expand by about 5%, about 10%, about 100%, about 400%, or about 500%. Local expansion of the core 40 within the fiber end 48 may take place via laser heating, plasma heating, resistance heating, or flame heating a portion of an optical fiber and allowing sufficient time to pass for a portion of the core 40 to diffuse into the cladding 34. Expansion of the core 40 may have an axial length along the optical fiber 18 of about 1 millimeter to about 5 millimeters, and in a specific example of about 4 millimeters. Expanding the core 40 and angling the fiber end 48 of the optical fiber 18 may decrease the back reflection from about −10 dB to about −350 dB, and in specific examples to below about −80 dB, −90 dB, −100 dB, −110 dB, −120 dB and below about −130 dB. Additionally or alternatively, the core 40 of the fiber end 48 may be expanded and positioned at locations other than at the face 150 of the ferrule 62. For example, a second optical fiber having similar dimensions to that of the expanded core 40 fiber end 48 may be positioned in the aperture 98 of the ferrule 62 and be optically coupled to the fiber end 48. In such embodiments, the optical coupling may take place at any point along the aperture 98 (e.g., inside the ferrule 62) as well as at the entrance to the aperture 98. The second optical fiber may then have an angled end, from which the electromagnetic beam 52 exits, to reduce back reflection.

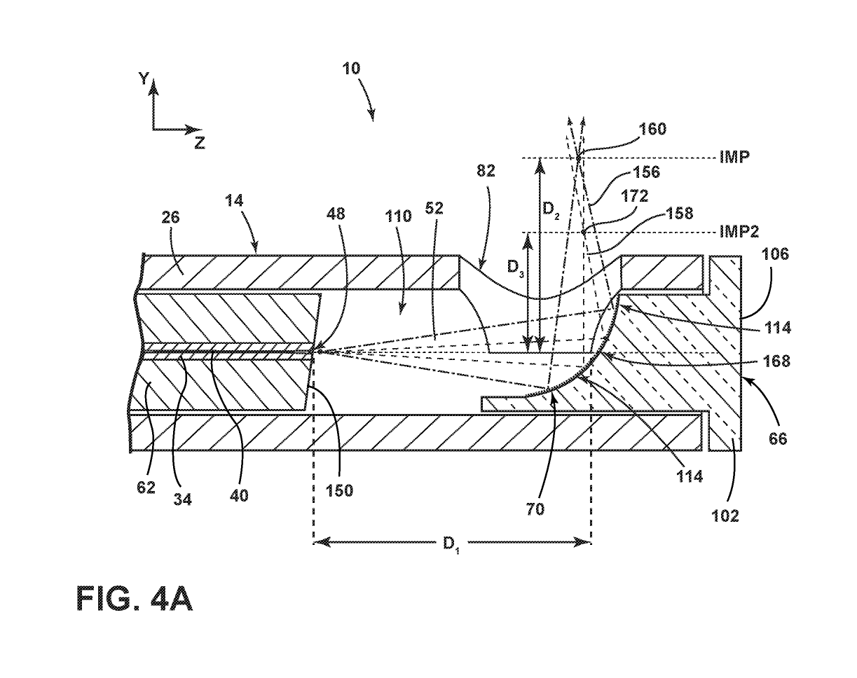

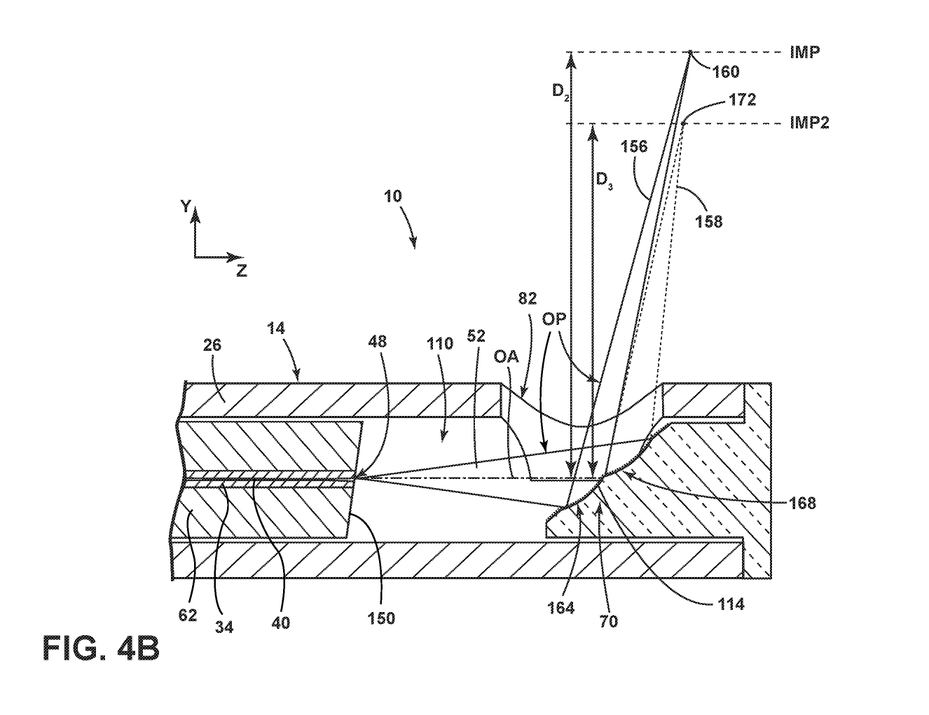

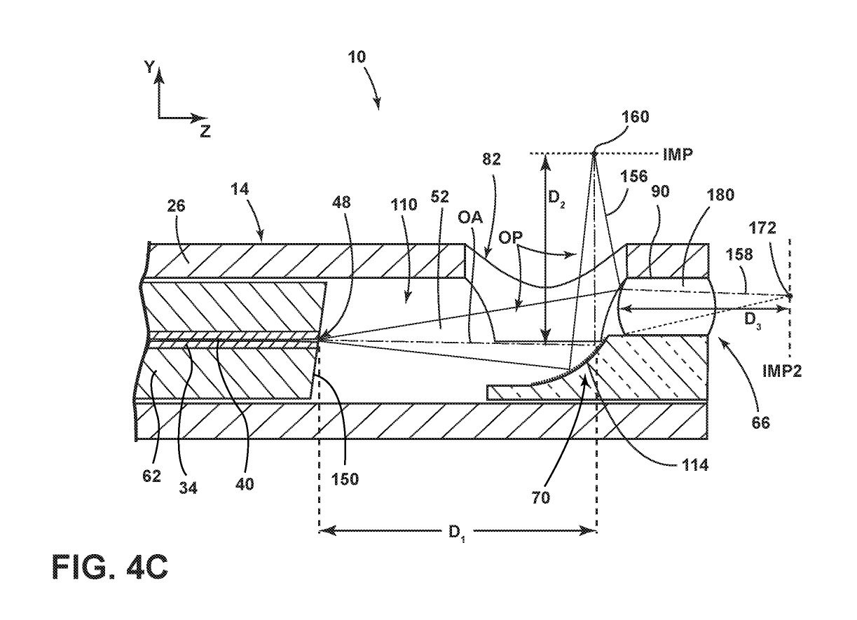

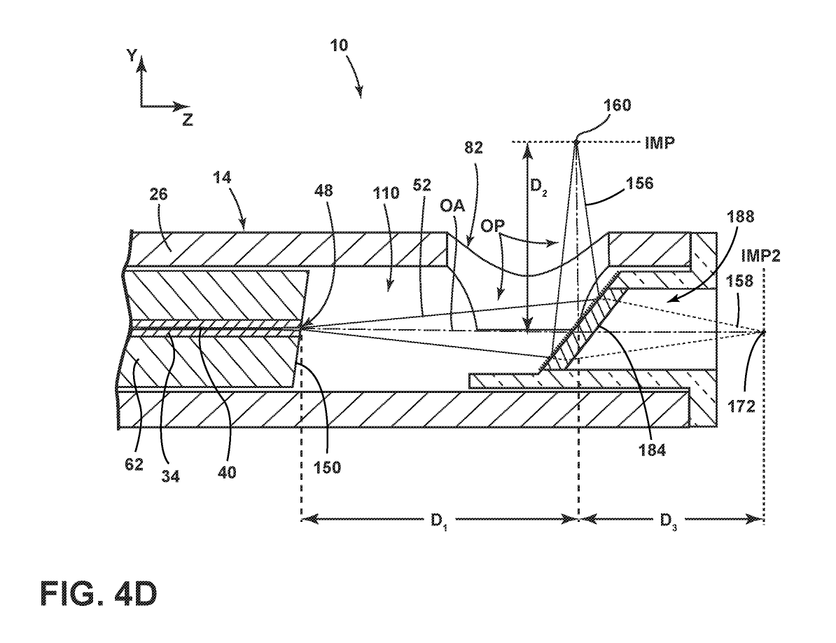

Referring now to FIGS. 4A-D, the beam-shaping insert 66 of the optical probe 10 may take a variety of configurations which form a second image spot 172 at a second image plane IMP2 having a second working distance D₃ away. The second working distance D₃ may be between about 1.0 millimeters and about 20.0 millimeters. In such an embodiment, the electromagnetic beam 52 may be split into a first portion 156 which forms the image spot 160 and a second portion 158 which forms the second image spot 172. In side-viewing embodiments (FIGS. 4A and 4B), both the first and second portions 156, 158 of the electromagnetic beam 52 may be directed to the side of the sheath 14 such that the second image spot 172 may be formed to a side of the optical probe 10 similar to that of the image spot 160. Such embodiments may be advantageous in that multiple locations of a sample being tested by the optical probe 10 may be in focus simultaneously, allowing a depth of the sample to be perceived. In forward-viewing embodiments (FIGS. 4C and 4D), the first portion 156 of the beam 52 may be directed to the side of the probe 10 to form image spot 160 and the second portion of the beam 158 may be directed along the Z-direction to form the second image spot 172 at the second image plane IMP2 forward of the probe 10. Such embodiments may be advantageous in that sample material in front of and to the side of the optical probe 10 may be scanned simultaneously, thus allowing an operator of the optical probe 10 greater flexibility in how to position the probe 10 relative to the sample. All of the depicted embodiments of FIGS.4A-D allow for the simultaneous formation of the image spot 160 and the second image spot 172, but may also allow selective formation of the image spot 160 and second image spot 172. It will be understood that elements of the depicted embodiments in FIGS. 4A-D may be combined with one another without departing from the spirit of this disclosure (e.g., forming multiple image spots to a side of the optical probe 10 while retaining forward viewing or forming multiple image spots forward of the optical probe 10).

Referring now to the depicted embodiment of FIGS. 4A and 4B, the beam-shaping element 70 may be configured as a dual zone reflector. In such an embodiment, the beam-shaping element 70 may define a first reflection zone 164 and a second reflection zone 168. In the embodiment of FIG. 4A, the first reflection zone 164 is depicted as encircling the second reflection zone 168, but the first and second reflection zones 164, 168 may take a variety of positional configurations. For example, FIG. 4B depicts the first reflection zone 164 above the second reflection zone 168. In yet other embodiments, the first and second reflection zones 164, 168 may be in a side by side configuration. The curved surface 118 may have a different conic constant or radius of curvature for each of the reflection zones 164, 168. The different conic constants and curvature radii allow the first reflection zone 164 to form the image spot 160 at the image plane IMP the working distance D2 away from the first portion 156 of the beam 52, while the second reflection zone 168 forms the second image spot 172 at the second image plane IMP2 the second working distance D₃ away from the second portion 158 of the beam 52. The image spot 160 and the second image spot 172 are depicted as being formed above one another, but may also be formed at the same image plane in a side by side configuration. The relative sizes of the first reflection zone 164 and the second reflection zone 168 may be different such that a greater portion of the electromagnetic beam 52 is captured by either of the first reflection reflective portion 174 or the refractive portion 176 and a more intense image spot (e.g., image spot 160 or the second image spot 172) may be formed from the corresponding portion.

Referring now to the depicted embodiment of FIG. 4C, the beam-shaping insert 66 includes a lens 180 in addition the beam-shaping element 70. The lens 180 may be integrally formed within the beam-shaping element 66, or maybe a separate structure configured to mate with the beam-shaping element 66 and the inner wall 90. Additionally or alternatively, the lens 180 may be positioned within the beam-shaping insert 66 such that it protrudes through the curved surface 118 and reflective element 114. The lens 180 may be a gradient index lens, a diffractive optical element, a Fresnel lens, and/or a refractive element such as that described above. As the electromagnetic beam 52 contacts the beam-shaping insert 66, the second portion 158 of the beam 52 passes through the lens 180 and exits the optical probe 10 to form the second image spot 172 at the second image plane IMP2 the second working distance D ₃ away. In optical coherence tomography applications of the optical probe 10, a computer which analyzes a signal from the optical probe 10 can distinguish between the data of the first image spot 160 and the second image spot 170 based on a time difference in the signal due to the different lengths of the working distances D2 and D3 of the image spot 160 and the second image spot 172.

Referring now to the depicted embodiment of FIG. 4D, the beam-shaping insert 66 includes a beam splitter 184 configured to reflect and focus the first portion 156 portion of the electromagnetic beam 52 while simultaneously refracting and focusing the second portion 158 of the electromagnetic beam 52. The beam-splitter 184 may be a dichroic lens, a polarization beam splitter, a half-silvered mirror, or any other form of beam splitter. The beam-splitter 184 may be altered to have a predetermined reflection vs refraction ratio, including 10/90, 20/80, 30/70, 40/60, 50/50, 60/40, 70/30, 80/20, 90/10 or smaller subdivisions thereof. By altering the ratio of reflection to refraction, the intensity of the image spot 160 and the second image spot 172 can be changed. The beam-splitter 184 may be integrally formed by the beam-shaping insert 66 (e.g., via half silvering of a clear polymeric embodiment of the beam-shaping insert 66) or may be mounted to the beam-shaping insert 66. In the depicted embodiment, the beam-shaping insert 66 defines a passage 188 through which the second portion 158 of the emitted beam 52 passes in order to form the second image spot 172 forward of the optical probe 10.

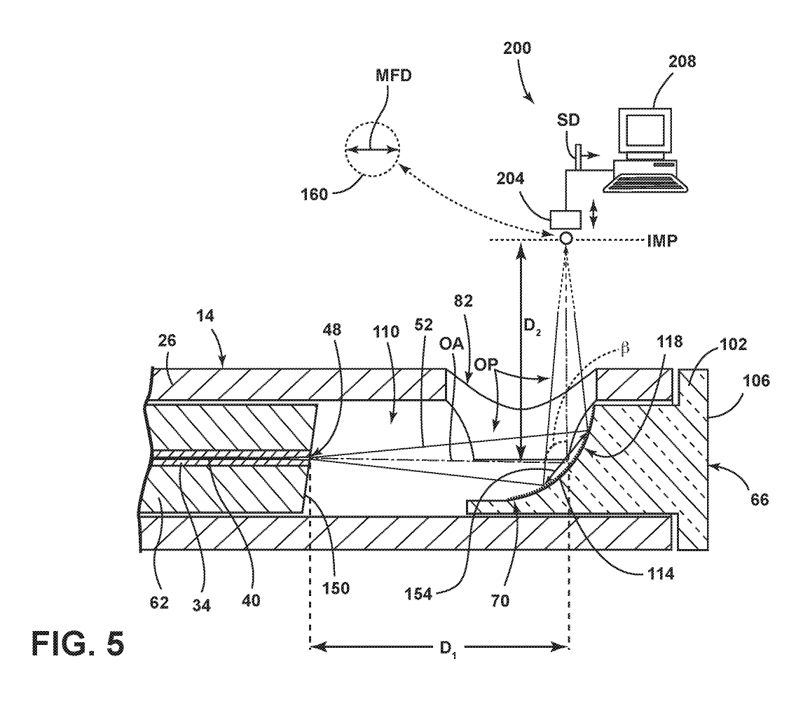

Referring now to FIG. 5, the optical probe 10 is depicted in use within an OCT alignment system 200. As explained above, light traveling within the optical fiber 18 exits the fiber end 48 and is emitted as beam 52 along the optical axis OA. The optical path OP of the beam 52 diverges as it passes through the gap 110 until it enters the beam-shaping element 70 and reflects from the reflective element 114. The curvature of the beam-shaping element 70 causes the light to converge uniformly to the image spot 160 due to the curved surface 118 being conic. In the depicted embodiment, as the beam 52 converges, it passes through the window 82 of the sheath 14 and forms the image spot 160 at the image plane IMP. The working distance D₂ is measured between the horizontal portion of the optical axis OA of the probe and the image plane IMP and may be between about 1 millimeter and about 20 millimeters.

The proper orientation of the optical probe 10 during manufacturing is facilitated by the use of the ferrule 62, the beam-shaping insert 66, and the OCT alignment system 200. In an exemplary method for alignment of the optical fiber 18, a photo detector 204 (e.g., camera or a rotating slit) can be used to capture at least one image of image spot 160 and generate a detector signal SD representative of the captured image. The captured image(s) can be analyzed, e.g., via a computer 208 that is operably connected to photodetector 204. The computer 208 can be used to analyze and display information about the captured image spot(s) 160. In an example, a plurality of image spots 160 are detected and compared to a reference spot (e.g., as obtained via optical modeling based on the design of the optical probe 10) to assess performance. If the detected image spots 160 are incorrect, an operator assembling the optical probe 10 may adjust a distance in the Z direction between the first and second portions 22, 26 of the sheath 14, or use the markings on the forward surface 106 of the beam-shaping insert 66, to adjust its orientation relative to the sheath 14. The use of the ferrule 62 and the beam-shaping insert 66 allow for near precise alignment of the optical probe 10 upon initial assembly.

The mode field diameter MFD is a measure of the spot size or beam width of light propagating in a single mode fiber or at another location in an optical system. The mode field diameter MFD within an optical fiber is a function of the source wavelength, fiber core radius and fiber refractive index profile. In the depicted embodiment, the optical probe 10 is capable of producing an image spot 160 having a mode field diameter MFD of between about 20 microns to about 100 microns at a 1/e² threshold at the image plane IMP. In a specific embodiment, the mode field diameter MDF may be about 22 microns. An exemplary mode field diameter of the optical fiber 18 may be 9.2 microns at a 1/e² threshold. The mode field diameter MFD may be sensed as an indicator of the quality of the image spot 160.

The position of optical fiber 18 can be axially adjusted within the optical probe 10 (e.g., by adjusting the first and second portions 22, 26 or moving the ferrule 62 or beam-shaping insert 66) based on making one or more measurements of image spot 160 until an acceptable or optimum image spot 160 is formed. In an example, the one or more measured image spots 160 are compared to a reference image spot or a reference image spot size. The ferrule 62 and the beam-shaping insert 66 can then be fixed in their respective aligned positions and orientations within the sheath 14 via one or more attachment methods (e.g., set screws, epoxies, adhesives, UV curable adhesives, friction fit, etc.).

In an exemplary embodiment of optical probe 10, the beam-shaping element 26 has an X-axis radius of curvature of about 1.16 millimeters and an X-axis conic constant of about 0.5858 and a Y-axis radius of curvature of about 1.2935 millimeters and a Y-axis conic constant of about 0.8235. Further, the conic shape of the beam-shaping element 70 is decentered along the Y-axis by about 0.7 millimeters, decentered along the Z-axis by about 0.089 millimeters, and has a rotation between the Y- and Z-axes of about 89.7°. The distance D₁ between the fiber end 40 and reflective element 114 is about 1.314 millimeters. Such an optical probe is capable of forming the image spot 160 at a working distance D2 of about 9.0 millimeters with a mode field diameter MFD of about 64 microns at the 1/e² threshold.

Because optical probe 10 and the exemplary optical coherence tomography alignment system 200 has a beam-shaping insert 66 which defines a reflective beam-shaping element 70, the system has no need for the use of spacers, GRIN lenses or refractive elements, such as lenses. Further, eliminating the use of multiple optical components is beneficial because there are fewer material interfaces which may result in optical back reflections or vignetting of the image spot 160. Additionally, by shaping the beam 52 into the image spot 160 solely based on reflection, higher power light sources may be used than conventional optical probes. Optical probes utilizing polymers as a refractive element are limited in the intensity of light they may refract; however, reflective systems do not have such limitations.

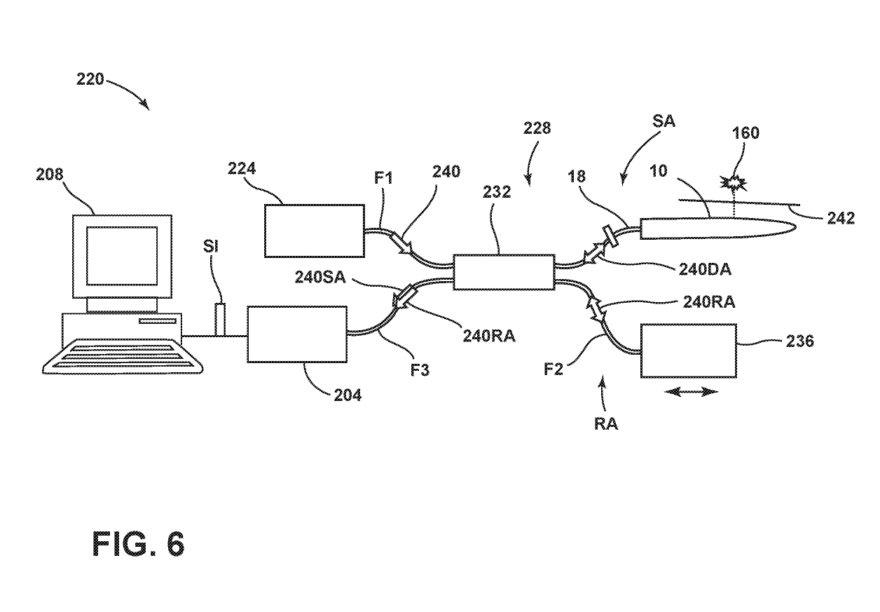

FIG. 6 illustrates an exemplary OCT system 220 that includes an embodiment of the optical probe 10 as disclosed herein. OCT system 220 includes a light source 224 and an interferometer 228. The light source 224 is optically connected to a fiber optic coupler (“coupler”) 232 via a first optical fiber section FI. OCT probe 10 is optically connected to coupler 232 via optical fiber 18 and constitutes the sample arm SA of the interferometer 228. OCT system 220 also includes a movable mirror system 236 optically connected to coupler 232 via an optical fiber section F2. Mirror system 236 and optical fiber section F2 constitute a reference arm RA of the interferometer 228. Mirror system 236 is configured to alter the length of the reference arm, e.g., via a movable mirror (not shown). OCT system 220 further includes the photodetector 204 optically coupled to coupler 232 via a third optical fiber section F3. Photodetector 204 in turn is electrically connected to computer 208.

In operation, light source 224 generates light 240 that travels to interferometer 228 over optical fiber section FI. The light 240 is divided by coupler 232 into light 240RA that travels in reference arm RA and light 240SA that travels in sample arm SA. The light 240RA that travels in reference arm RA is reflected by mirror system 236 and returns to coupler 232, which directs the light to photo detector 204. The light 240SA that travels in sample arm SA is processed by optical probe 10 as described above (where this light was referred to as just emitted beam 52) to form image spot 160 on or in a sample 244. The resulting scattered light is collected by optical probe 10 and directed through optical fiber 18 to coupler 232, which directs it (as light 240SA) to photodetector 204. The reference arm light 240RA and sample arm light 240SA interfere and the interfered light is detected by photodetector 204. Photodetector 204 generates an electrical signal SI in response thereto, which is then sent to computer 208 for processing using standard OCT signal processing techniques.

The optical interference of light 240SA from sample arm SA and light 240RA from reference arm RA is detected by photodetector 204 only when the optical path difference between the two arms is within the coherence length of light 240 from light source 224. Depth information from sample 244 is acquired by axially varying the optical path length of reference arm RA via mirror system 236 and detecting the interference between light from the reference arm and scattered light from the sample arm SA that originates from within the sample 244. A three-dimensional image is obtained by transversely scanning in two dimensions the optical path in the sample arm SA. The axial resolution of the process is determined by the coherence length.

It should be understood that although the use of the optical probe 10 was described in connection with only one OCT technique, the optical probe 10 may be used in a wide variety of applications, including other OCT techniques (e.g., Frequency Domain OCT, Spectral Domain OCT).

### EXAMPLES

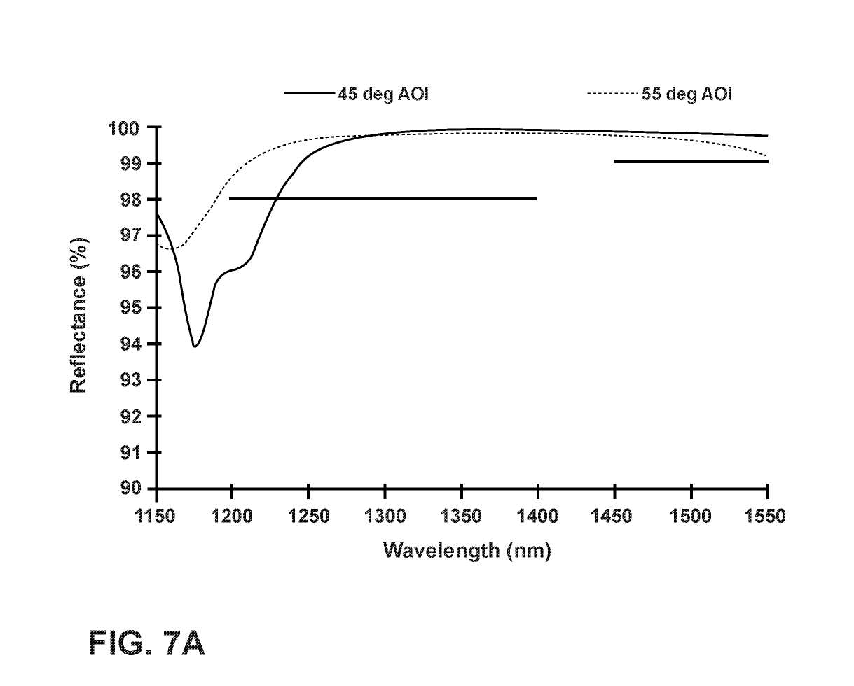

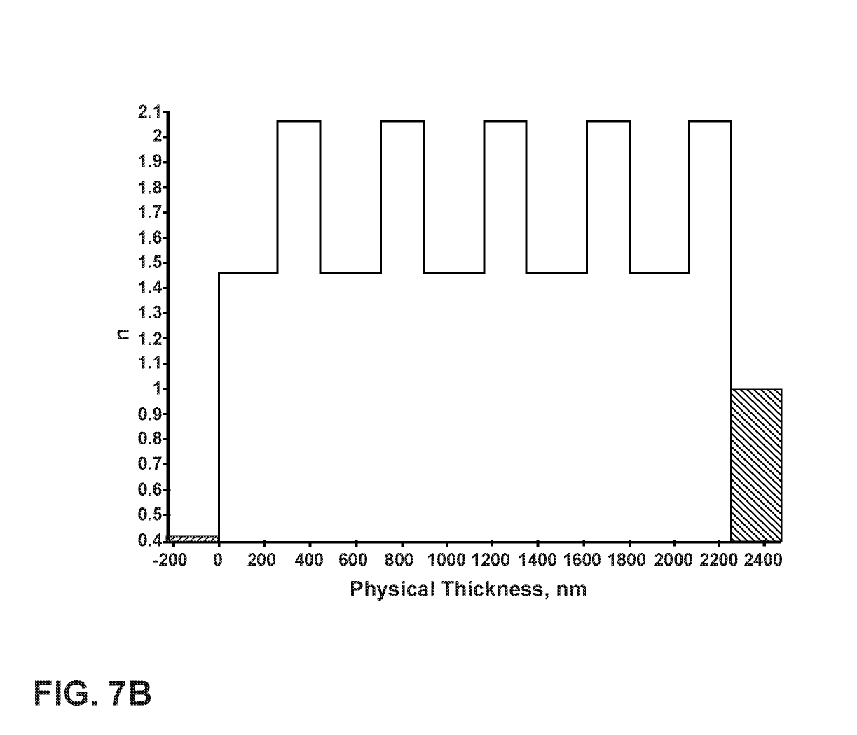

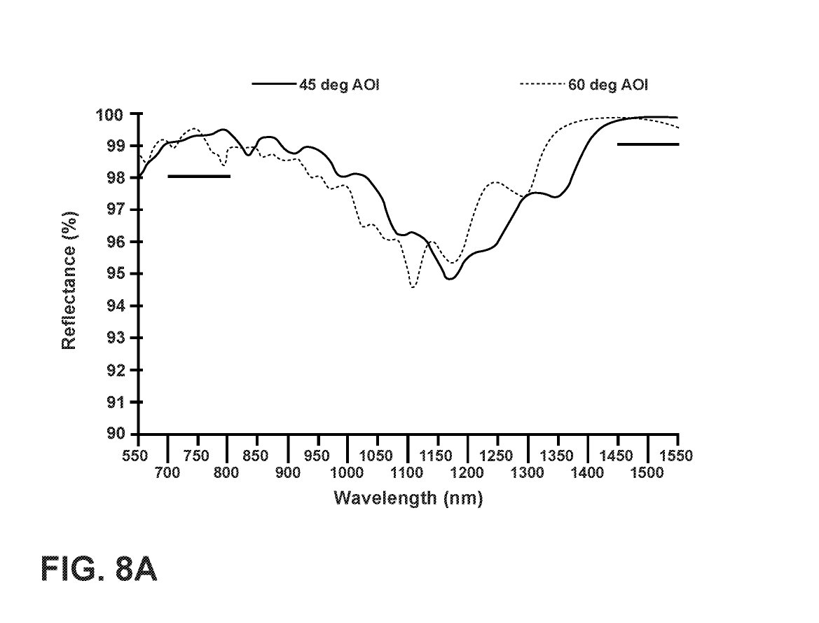

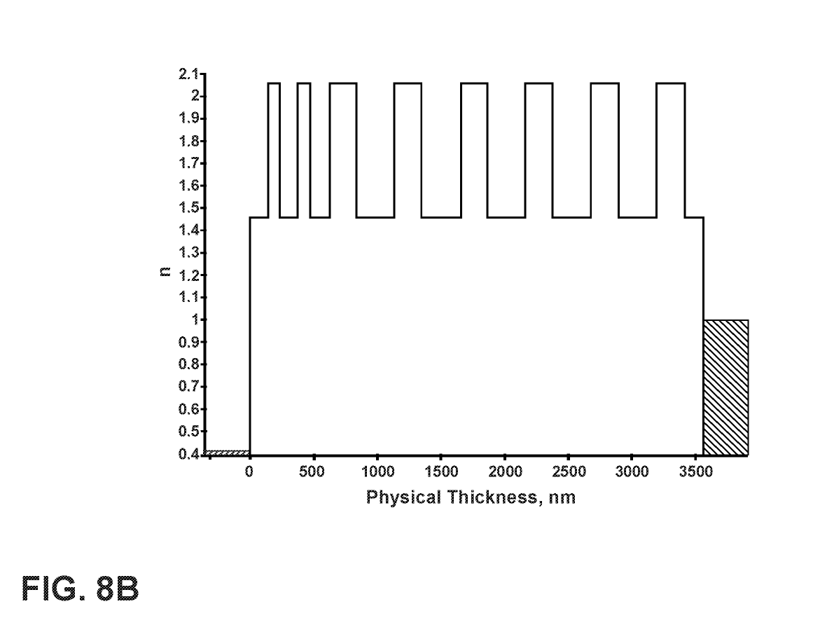

FIGS. 7A-8B are graphs and charts depicting computed data about specific examples of the reflective element 114 as made according to various aspect of this disclosure. FIGS. 7A-B correspond to a dual-channel mirror (e.g., reflective element 114) having a reflectance greater than about 98% for two different wavelength bands light (e.g., electromagnetic beam 52). FIG. 7A depicts a graph showing that the dual-channel mirror has a reflectance greater than about 98% at an angle of incidence of about 55° over a first wavelength band from about 1200 nanometers to about 1400 nanometers and a second wavelength band of from about 1450 nanometers to about 1550 nanometers. FIG. 7B depicts that the dual-channel mirror has a single dielectric stack (e.g., first dielectric stack 130) of alternating dielectric materials (e.g., the first dielectric layer and the second dielectric layer 130A, 130B), the layers having alternating thicknesses. In this example, the dielectric materials are SiO₂ and Ta₂O₅, with the SiO₂ layers having a refractive index n of 1.47 and the Ta₂O₅ layers having a refractive index n of about 2.06.

FIGS. 8A and 8B also depicts a dual channel mirror (e.g., reflective element 114) having a reflectance greater than about 98% for two separate wavelength bands of a light source (e.g., electromagnetic beam 52). FIG. 8A depicts a graph showing that the dual-channel mirror has a reflectance greater than about 98% over a first wavelength band from about 700 nanometers to about 800 nanometers and a second wavelength band of from about 1450 nanometers to about 1550 nanometers. FIG. 8B depicts that the dual-channel mirror has a two dielectric stacks (e.g., first dielectric stack 130 and second dielectric stack 134) of alternating dielectric materials (e.g., the first, second, third and fourth dielectric layers 130A, 130B, 134A, 134B), the stacks being separated based on dielectric layer thicknesses. In this example, the dielectric materials are SiO₂ and Ta₂O₅, with the SiO₂ layers having a refractive index n of 1.47 at 750 nanometers and the Ta₂O₅ layers having a refractive index n of about 2.06 at 1480 nanometers.

While the embodiments disclosed herein have been set forth for the purpose of illustration, the foregoing description should not be deemed to be a limitation on the scope of the disclosure or the appended claims. It will be apparent to those skilled in the art that various modifications and variations can be made without departing from the spirit or scope of the claims.

It will be understood by one having ordinary skill in the art that construction of the described invention and other components is not limited to any specific material. Other exemplary embodiments of the invention disclosed herein may be formed from a wide variety of materials, unless described otherwise herein. In this specification and the amended claims, the singular forms “a,” “an,” and “the” include plural reference unless the context clearly dictates otherwise.

Where a range of values is provided, it is understood that each intervening value, to the tenth of the unit of the lower limit, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated. It will be apparent to those skilled in the art that various modifications and variations can be made without departing from the spirit or scope of the claims.

1. A beam-shaping optical system suitable for use with optical coherence tomography, comprising:
a beam-shaping insert comprising a polymeric material, the beam-shaping insert integrally defining a beam-shaping element, wherein the beam-shaping element comprises a reflective element positioned on a curved surface;
a light source generating an electromagnetic beam; and
an optical fiber having a core and a cladding, the optical fiber having first end optically coupled to the light source and a fiber end configured to emit the electromagnetic beam toward the beam-shaping element,
wherein the reflective element has a reflectivity greater than about 98% for both a first wavelength band of the electromagnetic beam and a second wavelength band of the electromagnetic beam.

2. The beam-shaping optical system of claim 1, wherein the first wavelength band has a wavelength range of about 700 nanometers to about 800 nanometers and the second wavelength band has a wavelength range of about 1450 nanometers to about 1550 nanometers.

3. The beam-shaping optical system of claim 1, wherein the beam-shaping element further comprises a barrier layer positioned between the reflective element and the curved surface, the barrier layer comprising at least one of chromium, aluminum, and alumina.

4. The beam-shaping optical system of claim 3, wherein the barrier layer comprises a chromium layer, an aluminum layer, and an alumina layer.

5. The beam-shaping optical system of claim 4, wherein the chromium layer, aluminum layer, and alumina layer each have a thickness in the range of about 10 nanometers to about 60 nanometers.

6. The beam-shaping optical system of claim 1, wherein the reflective element comprises at least one dielectric stack including alternating layers of SiO₂ and at least one of Ta₂O₅, NbO₅, TiO₂, and HfO₂.

7. The beam-shaping optical system of claim 1, wherein the polymeric material of the beam-shaping body has a glass transition temperature greater than about 150° C.

8. The beam-shaping optical system of claim 1, wherein the electromagnetic beam has a peak intensity greater than about 1,000 watts/cm² as measured at the beam-shaping element when operating in the second wavelength band.

9. The beam-shaping optical system of claim 1, wherein the first and second wavelength bands are separated by at least 50 nanometers in wavelength.

10. An optical coherence tomography probe, comprising:
a sheath defining a central cavity;
a beam-shaping insert positioned in the central cavity, the insert comprising a polymeric material and defining a curved surface;
a reflective element positioned on the curved surface, the reflective element comprising:
a barrier layer comprising at least one layer of aluminum, chromium or alumina positioned on the curved surface,
a metal layer positioned on the barrier layer, and
at least one stack of alternating dielectric materials positioned on the metal layer;
a ferrule positioned within the central cavity; and
an optical fiber, the fiber supported by the ferrule including a fiber end configured to emit an electromagnetic beam toward the reflective element.

11. The optical coherence tomography probe of claim 10, wherein the electromagnetic beam has a peak intensity greater than about 500 watts/cm² as measured at the beam-shaping element.

12. The optical coherence tomography probe of claim 10, wherein the barrier layer comprises a chromium layer, an aluminum layer, and an alumina layer.

13. The optical coherence tomography probe of claim 12, wherein each of the chromium, aluminum, and alumina layers has a thickness of between about 10 nm and about 50 nm.

14. The optical coherence tomography probe of claim 10, wherein the at least one stack of alternating dielectric materials comprises alternating layers of SiO₂ and at least one of Ta₂O₅, NbO₅, TiO₂, and HfO₂.

15. The optical coherence tomography probe of claim 14, wherein the reflective element has a reflectivity greater than about 98% for both a first wavelength band of the electromagnetic beam and a second wavelength band of the electromagnetic beam.

16. The beam-shaping optical system of claim 15, wherein the first and second wavelength band s are separated by at least 50 nanometers in wavelength.

17. A method of forming an optical coherence tomography probe, comprising the steps:
forming a polymeric beam-shaping insert defining a curved surface;
depositing a barrier layer on the curved surface, the barrier layer comprising at least one layer of chromium, aluminum, and alumina;
depositing a metallic layer on the barrier layer; and
depositing a dielectric stack on the metallic layer to form a reflective element, wherein the reflective element is configured to reflect greater than about 98% of both a first wavelength band of an electromagnetic beam and a second wavelength band of an electromagnetic beam.

18. The method of forming an optical coherence tomography probe of claim 17, wherein the first wavelength band is an imaging band and the second wavelength band is a high power band.

19. The method of forming an optical coherence tomography probe of claim 17 or 18, wherein the dielectric stack comprises alternating layers of SiO₂ and at least one of Ta₂O₅, NbO₅, TiO₂, and HfO₂.

20. The method of forming an optical coherence tomography probe any of claim 17, further comprising the step of:
depositing a second dielectric stack adjacent the dielectric stack.

21. The method of forming an optical coherence tomography probe any of claim 17, wherein the polymer of the beam-shaping insert has a glass transition temperature greater than about 150° C.

22. The method of forming an optical coherence tomography probe of claim 17, wherein the electromagnetic beam has a peak intensity greater than about 1,000 watts/cm² as measured at the beam-shaping element when operating in the second wavelength band.

23. A beam-shaping optical system suitable for use with optical coherence tomography, comprising:
a sheath defining a central cavity;
a beam-shaping insert having a first beam-shaping element and a second beam-shaping element, the insert positioned within the cavity; and
an optical fiber having a core and a cladding disposed within the central cavity, the optical fiber having a fiber end configured to emit an electromagnetic beam toward the beam-shaping insert,
wherein the first beam-shaping element reflects a first portion of the electromagnetic beam and the second beam-shaping element refracts a second portion of the electromagnetic beam.

24. The beam-shaping optical system of claim 23, wherein the first portion of the electromagnetic beam is reflected and the second portion of the electromagnetic beam is refracted simultaneously.

25. The beam-shaping optical system of claim 24, wherein the first portion of the electromagnetic beam is reflected to a side of the sheath and the second portion of the electromagnetic beam is refracted forward of the sheath.

26. The beam-shaping optical system of claim 24, wherein the first portion of the electromagnetic beam is reflected to a side of the sheath and the second portion of the electromagnetic beam is reflected to the side of the sheath.

27. The beam-shaping optical system of claim 26, wherein the first beam-shaping element comprises a reflective element positioned on a curved surface integrally defined by the beam-shaping insert.

28. The beam-shaping optical system of claim 27, wherein the first beam-shaping element further comprises a barrier layer positioned between the reflective element and the curved surface having at least one layer of chromium, aluminum, and alumina.

29. The beam-shaping optical system of claim 23, wherein the first and second beam-shaping elements converge the electromagnetic beam to respective first and second image points, the first and second image points having different working distances.

30. The beam-shaping optical system of claim 23, wherein the second beam-shaping element comprises a lens.

31. An optical coherence tomography probe, comprising:
a sheath defining a central cavity;
a beam-shaping insert positioned near an end of the central cavity;
a beam-shaping element positioned on the beam-shaping insert; and
an optical fiber having a core and a cladding disposed within the central cavity, the optical fiber having a fiber end configured to emit an electromagnetic beam toward the beam-shaping element,
wherein the beam-shaping element is configured to focus a first portion of the electromagnetic beam to a side of the sheath and focus a second portion of the electromagnetic beam forward of the sheath.

32. The optical coherence tomography probe of claim 31, wherein the first portion of the electromagnetic beam is reflected and the second portion of the electromagnetic beam is refracted.

33. The optical coherence tomography probe of claim 32, wherein the first portion of the electromagnetic beam and the second portion of the electromagnetic beam are focused simultaneously.

34. The optical coherence tomography probe of claim 31, wherein the first and second beam-shaping elements converge the electromagnetic beam to respective first and second image points, the first and second image points having different working distances.

35. The optical coherence tomography probe of claim 31, wherein the beam-shaping element is one of a dichroic lens and a polarization beam splitter.

36. The optical coherence tomography probe of claim 31, further comprising a ferrule positioned within the sheath, wherein the optical fiber is positioned within the ferrule.

37. The beam-shaping optical system of claim 36, wherein the fiber end is prepared at an angle between about 4° and about 10°.

38. A method of forming multiple image spots, comprising the steps:
positioning an optical fiber having a core and a cladding within a ferrule;
positioning the ferrule within a central cavity of a sheath; and
emitting an electromagnetic beam from a fiber end of the optical fiber toward a beam-shaping insert, wherein the beam-shaping insert is configured to form a first image point at a first image plane and a second image point at a second image plane, the image planes being different working distances from the beam-shaping insert.

39. The method of forming multiple image spots of claim 38, wherein the beam-shaping insert comprises a first beam-shaping element and a second beam-shaping element.

40. The method of forming multiple image spots of claim 38, wherein the electromagnetic beam passes through an air gap between the fiber end and the first and second beam-shaping elements.

41. The method of forming multiple image spots of claim 38, wherein the beam-shaping insert includes a single beam-shaping element.

42. The method of forming multiple image spots of claim 40, wherein the first and second image spots are formed simultaneously.

43. The beam-shaping optical system of claim 1, wherein the electromagnetic beam has a peak intensity greater than about 500 watts/cm² as measured at the beam-shaping element when operating in the second wavelength band.
